(19) United States
(12) Patent Application Publication
(10) Pub. No.: US 2026/0355294 A1
(43) Pub. Date: Nov. 12, 2026

(54) BOTULINUM TOXIN FOR TREATING PERIPHERAL NEUROPATHIC PAIN

(71) Applicant: Merz Therapeutics GmbH, Frankfurt am Main (DE)

(72) Inventors: Irena Pulte, Frankfurt am Main (DE); Ilona Bicker, Frankfurt am Main (DE); Éric Viel, Nîmes (FR); Nadine Attal, Boulogne Billancourt (FR)

(21) Appl. No.: 19/486,840

(22) PCT Filed: May 23, 2024
(86) PCT No.: PCT/EP2024/064200

§ 371 Date: Nov. 21, 2025

(30) Foreign Application Priority Data
May 23, 2023 (EP) 23174933.4

(51) Int. Cl. A61K 38/48; A61P 25/02

(52) U.S. Cl. CPC A61K 38/4893; A61P 25/02; C12Y 304/24069

(57) ABSTRACT

The present invention relates to the use of botulinum toxin for the treatment of peripheral neuropathic pain (PNP) by local administration of botulinum toxin. The administration involves subcutaneous injection of an effective amount of botulinum toxin in the pain-affected skin area of a patient according to a specific administration regimen. The peripheral neuropathic pain can be, for example, post-herpetic neuralgia, peripheral nerve injury (e.g., due to surgery or trauma), trigeminal neuralgia, painful polyneuropathy (e.g., diabetic neuropathic pain), and painful radiculopathy.

### FIELD OF THE INVENTION

The present invention relates to the use of botulinum toxin for the treatment of peripheral neuropathic pain (PNP) by local administration of botulinum toxin. The administration involves subcutaneous injection of an effective amount of botulinum toxin in the pain-affected skin area of a patient according to a specific administration regimen. The peripheral neuropathic pain can be, for example, post-herpetic neuralgia, peripheral nerve injury (e.g., due to surgery or trauma), trigeminal neuralgia, painful polyneuropathy (e.g., diabetic neuropathic pain), and painful radiculopathy.

### BACKGROUND OF THE INVENTION

Pain is currently defined by the International Association for the Study of Pain (IASP) as “an unpleasant sensory and emotional experience associated with, or resembling that associated with, actual or potential tissue damage” (Raja et al., Pain, 2020, 161 (9):1976-1982). Among the different pain types, neuropathic pain (NP) is characterized by increased pain sensitivity and/or spontaneous pain and is defined by the IASP as “pain caused by a lesion or disease of the somatosensory nervous system” (Scholz et al., Pain, 2019, 160 (1):53-59). It may be experienced far from the nervous system lesion or disease (Raja et al, supra). Neuropathic pain may result from etiologically diverse disorders and the underlying pathophysiological mechanisms are complex and far from being fully understood (Scholz, supra). About 6-10% of adults are affected by chronic pain with neuropathic features (Kamerman et al., 2015, Pain, 156 (5):793-797), with detrimental consequences in terms of psychosocial wellbeing and health-related quality of life.

Depending on the location of the lesion or disease, neuropathic pain can be classified into peripheral neuropathic pain (PNP) and central neuropathic pain (CNP). PNP is defined as pain caused by a lesion or disease of the peripheral somatosensory nervous system, whereas CNP is defined as pain caused by a lesion or disease of the central somatosensory nervous system. The most common conditions of PNP include trigeminal neuralgia, peripheral nerve injury, painful polyneuropathy (e.g., diabetic neuropathic pain), postherpetic neuralgia, and painful radiculopathy. Conditions of central neuropathic pain include pain caused by spinal cord or brain injury, post-stroke pain, and pain associated with multiple sclerosis (Scholz et al., supra).

Neuropathic pain is challenging to treat due to its chronic course and the existing limitations of non-pharmacological and pharmacological approaches. An unmet medical need for new treatment options for neuropathic pain, in particular PNP, derives strongly from the current limitations of the mostly oral first-and second-line drug treatments. Many patients do not achieve sufficient pain relief with approved medications or do not tolerate the systemic drugs due to prevalent side effects such as dry mouth, nausea, or sleepiness, which often lead to discontinuation of therapy (Finnerup et al., Lancet Neurol., 2015, 14 (2):162-173). Furthermore, effective and safe non-opioid drug options for pain management are needed, as opioid misuse and addiction are an increasing public health challenge (Coussens et al., J. Pharmacol. Exp. Ther., 2019, 371 (2):396-408).

Local injection treatment with botulinum toxin (botulinum neurotoxin or BoNT) is a newly emerging treatment option. BoNT is a potent neurotoxin produced by Clostridium botulinum and its putative mechanism of action of BoNT in neuropathic pain might involve, among others, inhibition of the release of neurotransmitters and pain mediators (e.g., substance P, glutamate, and calcitonin gene related protein (CGRP)) and reduction of activity of the transient receptor potential (TRP) ion channels, involved in the transduction of noxious stimuli (Go et al., Front. Mol. Neurosci., 2021, 14:772719, doi: 10.3389/fnmol.2021.772719; J. H. Park and H. J. Park, Toxins, 2017, 9 (9):260). Several clinical trials with BoNT have been performed in the past years for treating peripheral neuropathic pain and showed promising efficacy and safety of BoNT treatment in several PNP conditions.

In a randomized, double-blind, two-arm, single-dose, placebo-controlled clinical trial performed by Apalla et al. (Clin. J. Pain., 2013, 29 (10):857-64), the efficacy and safety of BoNT Type A, Botox®, was evaluated in patients with postherpetic neuralgia. Each patient received 40 injections, each with 5 U of Botox®, resulting in the total maximum dose of 200 U. Injections were administered subcutaneously in a chessboard manner with a minimal distance between injection sites of 1 cm. It was concluded that BoNT Type A is a promising therapeutic modality for postherpetic neuralgia. Furthermore, Attal et al. (Lancet Neurol., 2016, 15 (6):555-65), in a randomized, double-blind, two-arm, two dose, placebo-controlled trial, investigated efficacy and safety of BoNT Type A, Botox®, in patients with peripheral neuropathic pain (different PNP conditions were included). The total dose of BoNT Type A, Botox®, was determined by the size of the painful area, without exceeding 60 injection sites (i.e., without exceeding a total dose of 300 U). Injections were administered subcutaneously, 0.2 mL (5 U) per injection site, with injection sites 1.5 to 2 cm apart. In conclusion, two administrations of BoNT Type A had a sustained analgesic effect against peripheral neuropathic pain.

Other PNP studies using botulinum toxin include the randomized double-blind placebo-controlled single-dose study of Ghasemi et al. (J. Res. Med. Sci., 2014, 19 (2):106-111), in which 40 participants with diabetic PNP in both feet were injected intradermally with BoNT Type A (Dysport®) into 12 injection points in a grid (3×4 injection points) on the dorsum of one foot. The authors concluded that BoNT Type A has a significant effect on diabetic peripheral neuropathic pain. Ranoux et al. (Ann. Neurol., 2008, 64 (3):274-283) reported a double-blind, placebo-controlled parallel group trial design study which investigated the use of BoNT Type A, Botox®, in 29 patients with focal painful neuropathies (e.g., post-traumatic/postoperative pain or postherpetic neuralgia) associated with mechanical allodynia. Botox® or placebo was injected intradermally into the skin 1.5 cm apart, without exceeding the predetermined maximum number of sites, which was fixed at 40 (corresponding to a maximal dose of 200 units). The authors concluded that intradermal injection of BoNT Type A has direct analgesic effects in patients with focal chronic neuropathic pain associated with allodynia.

In addition, in the randomized double-blind placebo-controlled single-dose study of Taheri et al. (Diabetes Metab. Syndr., 2020, 14 (6):1823-1828), it was found that intradermal injection of BoNT in patients with painful diabetic neuropathy, using injections in a 4×5 grid with a distance of 1 cm between them, is effective in reducing neuropathic pain and improving symptoms associated with neuropathic pain. Xiao et al. (Pain Med., 2010, 11 (12):1827-1833), in a prospective, randomized, placebo-controlled, double blind clinical trial conducted in China, demonstrated that subcutaneous injection of BoNT Type A (Lanzhou Institute of Biological Products, China) into the affected area of subjects with postherpetic neuralgia within 1.0 to 2.0 cm radius of skin using 1 mL volume (total dose of 77±18.5 U of BoNT Type A) significantly decreased pain in postherpetic neuralgia. Yuan et al. (Neurology, 2009, 72 (17):1473-1478) conducted a double-blind, placebo-controlled crossover trial in 18 patients to evaluate the effectiveness of intradermal BoNT Type A (Botox®) for diabetic neuropathic pain. In this trial, BoNT Type A, Botox®, was administered intradermally into each foot of a patient in a grid pattern (typically 2 to 3 cm apart covering a total of 12 sites), with each injection comprising approximately 4 U Botox®, in a volume of 0.1 mL. The authors found that BoNT Type A significantly reduced diabetic neuropathic pain and transiently improved sleep quality.

Furthermore, four randomized double-blind placebo-controlled single-dose studies investigated the effect of BoNT Type A, Botox® (Shehata et al., J. Headache Pain, 2013, 14 (1):92; Zuñiga et al., Clin. Neuropharmacol., 2013, 36 (5):146-150) and BoNT Type A (Lanzhou Institute of Biological Products, China) (Wu et al., Cephalagia, 2012, 32 (6):443-450; Zhang et al., J. Headache Pain, 2014, 15 (1):65) on trigeminal neuralgia. All studies demonstrated statistically significant superiority of BoNT/A over placebo for pain reduction and, if reported, also for pain attack frequency. However, in contrast to these positive clinical trials results, a multicenter, double-blind, placebo-controlled, parallel-group trial sponsored by Allergan, registered as NCT00168441 in ClinicalTrials. gov did not find a statistically significant difference of BoNT Type A (Botox®) treatment involving 2 to 80 intradermal injections (5 U to 200 U) versus placebo in patients with postherpetic neuralgia.

Although the results from the various clinical studies reflect the advantages of the minimally invasive, non-systemic therapy with BoNT compared to established approaches for neuropathic pain relief, there is no established dose, maximum dose, dilution, number of injection sites, or distance between the injection sites for any BoNT product in the treatment of neuropathic pain.

### OBJECT OF THE INVENTION

In view of the above, the object of the present invention is how to provide an improved treatment for peripheral neuropathic pain.

### SUMMARY OF THE INVENTION

The present invention relates to the use of botulinum toxin for the treatment of peripheral neuropathic pain. It was found that the use of a specific dosing and injection scheme for the administration of botulinum toxin is highly effective in the reduction of peripheral neuropathic pain while being well tolerable and safe.

In a first aspect, the present invention relates to a botulinum toxin for use in the treatment of peripheral neuropathic pain, wherein the botulinum toxin is subcutaneously injected into a skin area of a patient at a concentration of 35 U/ml to 65 U/ml, and at a dose of 4.0 U to 6.0 U per injection point and a volume of 0.07 ml to 0.15 ml per injection point, and wherein the distance between injection points is 1.5 cm to 2.5 cm.

The peripheral neuropathic pain is preferably selected from the group consisting of post-herpetic neuralgia (PHN), peripheral nerve injury (e.g., due to surgery or trauma), trigeminal neuralgia, painful polyneuropathy (e.g., diabetic neuropathic pain (DNP)), and painful radiculopathy. Within the present invention, the PNP is particularly preferred post-herpetic neuralgia (PHN), PNP due to peripheral nerve injury, in particular peripheral nerve injury due to surgery or peripheral nerve injury due to trauma, or painful polyneuropathy, in particular diabetic neuropathic pain (DNP).

In the context of the present invention, the botulinum toxin is preferably of type A. Further, the botulinum toxin may be in a form that is free of complexing proteins or is in the form of a complex that contains complexing proteins. Preferably, the botulinum toxin is of type A and in a form that is free of complexing proteins or, in an alternative preferred embodiment, is of type A and in the form of a complex that contains complexing proteins.

In a second aspect, the present invention relates to a method for the treatment of peripheral neuropathic pain, the method comprising administering an effective amount of botulinum toxin by subcutaneous injection into a skin area of a patient at a concentration of 35 U/ml to 65 U/ml, and at a dose of 4.0 U to 6.0 U per injection point and a volume of 0.07 ml to 0.15 ml per injection point, and wherein the distance between injection points is 1.5 cm to 2.5 cm.

Preferred embodiments are set forth in the appended dependent claims and in the following detailed description.

### DETAILED DESCRIPTION OF THE INVENTION

The present invention provides a specific dosing and injection scheme that results in beneficial treatment effects of BoNT injections on peripheral neuropathic pain. The BoNT dosing and injection scheme is not only highly efficient in reducing PNP and symptoms associated therewith but is also well tolerated and safe. The BoNT is locally injected into painful skin areas. It is a minimally invasive treatment modality that has only minimal side effects. Compared to commonly prescribed systemic drugs, the local injection treatment with BoNT does not have prevalent side effects (e.g., dry mouth, nausea and sleepiness) and does not involve the problem of opioid misuse and addiction. Thus, in terms of efficacy, safety, and tolerability, the specific local injection treatment with BoNT according to the present invention is a favourable therapeutic approach for the treatment of peripheral neuropathic pain.

The mentioned advantages are the result of a specific combination of different aspects of administration, including route of administration (subcutaneous injections), distance between injection sites (preferably 2 cm), dose of BoNT per injection site (preferably 5 U), concentration (preferably 50 U BoNT per 1 mL), total number of injection sites (maximum 90) and total dose (maximum 400 U). This unique local treatment regimen makes a significant contribution to the art by establishing a new treatment option in neuropathic pain management. The local treatment fills an important gap in this burdensome chronic condition, with a medical need for an effective therapeutic option without systemic side effects, in an era of opioid overuse, misuse, and addiction.

As used herein, the term “peripheral neuropathic pain” or “PNP” refers to pain caused by a lesion or disease of the peripheral somatosensory nervous system. It does not include central neuropathic pain (CNP) which is defined as pain caused by a lesion or disease of the central somatosensory nervous system. Causes of peripheral neuropathic pain include, for example, postherpetic neuralgia, peripheral nerve injury (e.g., due to surgery or trauma), trigeminal neuralgia, painful polyneuropathy (e.g., diabetic neuropathic pain (DNP)), and painful radiculopathy. Within the present invention, the PNP preferably is, or is caused by, post-herpetic neuralgia (PHN), PNP due to peripheral nerve injury (e.g., due to surgery or trauma), or painful polyneuropathy, in particular diabetic neuropathic pain (DNP).

The type of pain is not particularly limited and includes all symptoms of PNP. For example, some patients have allodynia, which is a symptom of PNP. Patients with allodynia feel pain from a stimulus that should not be painful (e.g., a patient who puts on a T-shirt and feels pain from the touch of the fabric on the skin, or a patient who washes their hands with lukewarm water and feels pain from it). However, allodynia is just one of the many symptoms of PNP. Other symptoms include, for example, “pins and needles”, a numb feeling, electric shocks etc. There are some assumptions that allodynia may be a symptom which is well susceptible to botulinum toxin treatment and, thus, any treatment of a PNP patient with allodynia is to be understood to include the treatment of allodynia.

The term “skin area of a patient” , as used herein, generally refers to the skin area afflicted with PNP, also referred to herein as “PNP area”, “PNP skin area”, “painful area”, “painful skin area” or “pain-affected skin area”. The location and size of the painful area to be treated are very individual and depend on the patient. In particular, the location and size of the painful area depend on the nerve that was damaged and the cause of PNP. However, for postherpetic neuralgia, although it can occur anywhere on the body, PNP often occurs on the thorax. Further, the areas associated with postherpetic neuralgia might be larger than for those with nerve injury. Diabetic PNP usually occurs first in both feet, but it can also appear in the hands and, when it progresses further, affect legs, arms and other body parts. Postsurgical/post-traumatic PNP occurs wherever the nerve injury occurred. As for assessing the PNP area, this is usually done by the treating physician asking the patient to outline the painful area. Patients can usually mark the painful area quite accurately.

The term “patient”, as used herein, is not particularly limited and generally refers to a human, who is in the need of pain treatment. Preferably, a “patient” in the context of the present invention is a human having PNP rated as at least possible by the NeuPSIG/IASP grading system (Finnerup et al., Neuropathic pain: an updated grading system for research and clinical practice Pain, 2016, 157 (8):1599-1606).

Herein, numerical values without decimal places shall be understood to include all numerical values with one or more decimal places that, applying common rules of rounding, give the numerical value without decimal places. For example, the numerical value 50 includes 49.5 or 49.50 (which are rounded up to 50) and 50.4 or 50.49 (which are rounded off to 50), and all numerical values in between. Likewise, numerical values with one decimal place shall be understood to include all numerical values with two or more decimal places that, applying common rules of rounding, give the numerical value with one decimal place (e.g., 3.95 and 4.04 are rounded up/off to 4.0). Also, numerical values with two decimal places shall be understood to include all numerical values with three or more decimal places that, applying common rules of rounding, give the numerical value with two decimal places (e.g., 0.085 and 0.094 are rounded up/off to 0.09). As regards endpoints of ranges, the same applies, e.g., a range of 35 to 65 includes the range of 34.5 to 65.4, a range of 1.5 to 2.5 includes the range of 1.45 to 2.54, and a range of 0.07 to 0.15 includes the range of 0.065 to 0.154.

The term “comprising”, like the terms “including” and containing”, and any variations thereof such as “comprises”, “includes” and “contains,”, are intended to refer to a non-exclusive inclusion, such that a process, method, product-by-process, composition or formulation that comprises, includes, or contains an element or list of elements does not include only those elements but can include other elements not expressly listed for such process, method, product-by-process, composition or formulation. In addition, within the framework of the present invention, it is intended that each of the terms “comprise,” “comprising”, “includes”, “including”, “contains”, “containing”, and any variations thereof, can be replaced by the term “consists” or “consisting”, or any variation thereof, which will be understood to refer to an exclusive inclusion of the elements indicated. Furthermore, the terms “a” and “an” and “the” and similar reference used in the context of the present invention are to be construed to cover both the singular and the plural and, thus, may also relate to “at least one” or “more than one”, unless otherwise indicated herein or clearly contradicted by the context.

In a first aspect, the present invention relates to a botulinum toxin for use in the treatment of peripheral neuropathic pain, wherein the botulinum toxin is subcutaneously injected into a skin area of a patient at a concentration of 35 U/ml to 65 U/ml, and at a dose of 4.0 U to 6.0 U per injection point and a volume of 0.07 ml to 0.15 ml per injection point, and wherein the distance between injection points is 1.5 cm to 2.5 cm.

According to the present invention, the injection volume per injection point is preferably 0.07 ml to 0.14 ml, more preferably 0.08 ml to 0.13 ml, still more preferably 0.08 ml to 0.12 ml, yet still more preferably 0.09 ml to 0.11 ml, and most preferably 0.10 ml. More specifically, the injection volume per injection point in accordance with the present invention may be within the range of 0.07 ml to 0.14 ml, and is preferably 0.07 ml to 0.13 ml, more preferably 0.07 ml to 0.12 ml, still more preferably 0.07 ml to 0.11 ml, or is preferably 0.08 ml to 0.15 ml, more preferably 0.08 ml to 0.14 ml, still more preferably 0.08 ml to 0.13 ml, yet still more preferably 0.08 ml to 0.12 ml, particularly preferably 0.08 ml to 0.11 ml, or is preferably 0.09 ml to 0.15 ml, more preferably 0.09 ml to 0.14 ml, still more preferably 0.09 ml to 0.13 ml, yet still more preferably 0.09 ml to 0.12 ml, and particularly preferably 0.09 ml to 0.11 ml.

The dose per injection point is preferably 4.5 U to 5.5 U. Thus, the dose per injection point may be, e.g., 4.6 U, 4.7 U, 4.8 U, 4.9 U, 5.0 U, 5.1 U, 5.2 U, 5.3 U, and 5.4 U. A particularly preferred dose per injection point is 5.0 U.

In a preferred embodiment of the first aspect of the invention, the concentration of botulinum toxin is within the range of 40 U/ml to 60 U/ml, for example 45 U/ml, 50 U/ml, and 55 U/ml, the dose is 4.0 U to 6.0 U per injection point and the volume is 0.07 ml to 0.15 ml per injection point. The volume per injection point is preferably 0.07 ml to 0.14 ml and more preferred injection volumes are as described above in connection with the first aspect of the invention. The “concentration”, as used herein, refers to the concentration of botulinum toxin in the liquid formulation that is administered by subcutaneous injection.

Preferably, in said preferred embodiment, the dose per injection point is 4.5 U to 5.5 U (including, e.g., 4.6 U, 4.7 U, 4.8 U, 4.9 U, 5.0 U, 5.1 U, 5.2 U, 5.3 U, 5.4 U), and most preferable 5.0 U) and the volume per injection point is 0.07 ml to 0.14 ml. Preferably, the volume per injection point is 0.08 ml to 0.13 ml, still more preferably 0.08 ml to 0.12 ml, yet still more preferably 0.09 ml to 0.11 ml, and most preferably 0.10 ml. More specifically, the volume per injection point is preferably 0.07 ml to 0.13 ml, more preferably 0.07 ml to 0.12 ml, still more preferably 0.07 ml to 0.11 ml, or is preferably 0.08 ml to 0.14 ml, more preferably 0.08 ml to 0.13 ml, still more preferably 0.08 ml to 0.12 ml, yet still more preferably 0.08 ml to 0.11 ml, or is preferably 0.09 ml to 0.14 ml, more preferably 0.09 ml to 0.13 ml, still more preferably 0.09 ml to 0.12 ml, and yet still more preferably 0.09 ml to 0.11 ml.

In accordance with the aspects and embodiments of the present invention, the total dose of the botulinum toxin is up to 400 U and/or the total number of injection points is up to 90. Preferably, the total dose is between 100 U and 400 U or up to 300 U or between 200 U and 300 U or from more than 300 U to 400 U. Preferably, the total number of injection points is 40 to 90 or 50 to 90 or up to 80 or 50 to 80 or 60 to 80.

As regards the injection pattern, the injection points are typically arranged such that the distance between injection points is 1.5 cm to 2.5 cm, preferably 1.6 cm to 2.4 cm, more preferably 1.7 cm to 2.3 cm, still more preferably 1.8 cm to 2.2 cm, yet still more preferably 1.9 cm to 2.1 cm, and most preferably 2.0 cm.

The expression “distance between injection points”, as used herein, means the distance between any given injection point and its neighbouring injection points. In particular, the expression “distance between injection points” refers to the distance between any given injection point and another injection point that is closest to said given injection point, unless otherwise stated.

In accordance with the present invention, exemplary preferred combinations of concentration, dose per injection point, volume per injection point, and distance between injection points include the following: (1) concentration of 35 U/ml to 65 U/ml, dose per injection point of 4.0 U to 6.0 U, volume per injection point of 0.08 ml to 0.13 ml, distance between injection points of 1.5 cm to 2.5 cm, (2) concentration of 35 U/ml to 65 U/ml, dose per injection point of 4.0 U to 6.0 U, volume per injection point of 0.09 ml to 0.12 ml, distance between injection points of 1.5 cm to 2.5 cm, (3) concentration of 35 U/ml to 65 U/ml, dose per injection point of 4.0 U to 6.0 U, volume per injection point of 0.09 ml to 0.11 ml, distance between injection points of 1.5 cm to 2.5 cm, (4) concentration of 35 U/ml to 65 U/ml, dose per injection point of 4.0 U to 6.0 U, volume per injection point of 0.07 ml to 0.15 ml, distance between injection points of 1.6 cm to 2.4 cm, (5) concentration of 35 U/ml to 65 U/ml, dose per injection point of 4.0 U to 6.0 U, volume per injection point of 0.07 ml to 0.15 ml, distance between injection points of 1.7 cm to 2.3 cm, (6) concentration of 35 U/ml to 65 U/ml, dose per injection point of 4.0 U to 6.0 U, volume per injection point of 0.07 ml to 0.15 ml, distance between injection points of 1.8 cm to 2.2 cm, (7) concentration of 35 U/ml to 65 U/ml, dose per injection point of 4.0 U to 6.0 U, volume per injection point of 0.08 ml to 0.13 ml, distance between injection points of 1.6 cm to 2.4 cm, (8) concentration of 35 U/ml to 65 U/ml, dose per injection point of 4.0 U to 6.0 U, volume per injection point of 0.09 ml to 0.12 ml, distance between injection points of 1.7 cm to 2.3 cm, (9) concentration of 35 U/ml to 65 U/ml, dose per injection point of 4.0 U to 6.0 U, volume per injection point of 0.09 ml to 0.11 ml, distance between injection points of 1.8 cm to 2.2 cm, (10) concentration of 35 U/ml to 65 U/ml, dose per injection point of 4.5 U to 5.5 U, volume per injection point of 0.07 ml to 0.15 ml, distance between injection points of 1.5 cm to 2.5 cm, (11) concentration of 35 U/ml to 65 U/ml, dose per injection point of 4.5 U to 5.5 U, volume per injection point of 0.08 ml to 0.13 ml, distance between injection points of 1.5 cm to 2.5 cm, (12) concentration of 35 U/ml to 65 U/ml, dose per injection point of 4.5 U to 5.5 U, volume per injection point of 0.08 ml to 0.12 ml, distance between injection points of 1.5 cm to 2.5 cm, (13) concentration of 35 U/ml to 65 U/ml, dose per injection point of 4.5 U to 5.5 U, volume per injection point of 0.09 ml to 0.11 ml, distance between injection points of 1.5 cm to 2.5 cm, (14) concentration of 40 U/ml to 60 U/ml, dose per injection point of 4.5 U to 5.5 U, volume per injection point of 0.07 ml to 0.15 ml, distance between injection points of 1.5 cm to 2.5 cm, (15) concentration of 40 U/ml to 60 U/ml, dose per injection point of 4.5 U to 5.5 U, volume per injection point of 0.08 ml to 0.13 ml, distance between injection points of 1.5 cm to 2.5 cm, (16) concentration of 40 U/ml to 60 U/ml, dose per injection point of 4.5 U to 5.5 U, volume per injection point of 0.08 ml to 0.12 ml, distance between injection points of 1.5 cm to 2.5 cm, (17) concentration of 40 U/ml to 60 U/ml, dose per injection point of 4.5 U to 5.5 U, volume per injection point of 0.09 ml to 0.11 ml, distance between injection points of 1.5 cm to 2.5 cm, (18) concentration of 35 U/ml to 65 U/ml, dose per injection point of 4.5 U to 5.5 U, volume per injection point of 0.07 ml to 0.15 ml, distance between injection points of 1.7 cm to 2.3 cm, (19) concentration of 35 U/ml to 65 U/ml, dose per injection point of 4.5 U to 5.5 U, volume per injection point of 0.08 ml to 0.13 ml, distance between injection points of 1.7 cm to 2.3 cm, (20) concentration of 35 U/ml to 65 U/ml, dose per injection point of 4.5 U to 5.5 U, volume per injection point of 0.08 ml to 0.12 ml, distance between injection points of 1.7 cm to 2.3 cm, (21) concentration of 35 U/ml to 65 U/ml, dose per injection point of 4.5 U to 5.5 U, volume per injection point of 0.09 ml to 0.11 ml, distance between injection points of 1.5 cm to 2.5 cm, (22) concentration of 35 U/ml to 65 U/ml, dose per injection point of 4.5 U to 5.5 U, volume per injection point of 0.07 ml to 0.15 ml, distance between injection points of 1.8 cm to 2.2 cm, (23) concentration of 35 U/ml to 65 U/ml, dose per injection point of 4.5 U to 5.5 U, volume per injection point of 0.08 ml to 0.13 ml, distance between injection points of 1.8 cm to 2.2 cm, (24) concentration of 35 U/ml to 65 U/ml, dose per injection point of 4.5 U to 5.5 U, volume per injection point of 0.08 ml to 0.12 ml, distance between injection points of 1.8 cm to 2.2 cm, (25) concentration of 35 U/ml to 65 U/ml, dose per injection point of 4.5 U to 5.5 U, volume per injection point of 0.09 ml to 0.11 ml, distance between injection points of 1.8 cm to 2.2 cm, (26) concentration of 40 U/ml to 60 U/ml, dose per injection point of 4.5 U to 5.5 U, volume per injection point of 0.07 ml to 0.15 ml, distance between injection points of 1.7 cm to 2.3 cm, (27) concentration of 40 U/ml to 60 U/ml, dose per injection point of 4.5 U to 5.5 U, volume per injection point of 0.08 ml to 0.13 ml, distance between injection points of 1.7 cm to 2.3 cm, (28) concentration of 40 U/ml to 60 U/ml, dose per injection point of 4.5 U to 5.5 U, volume per injection point of 0.08 ml to 0.12 ml, distance between injection points of 1.7 cm to 2.3 cm, (29) concentration of 40 U/ml to 60 U/ml, dose per injection point of 4.5 U to 5.5 U, volume per injection point of 0.09 ml to 0.11 ml, distance between injection points of 1.7 cm to 2.3 cm, (30) concentration of 40 U/ml to 60 U/ml, dose per injection point of 4.5 U to 5.5 U, volume per injection point of 0.07 ml to 0.15 ml, distance between injection points of 1.8 cm to 2.2 cm, (31) concentration of 40 U/ml to 60 U/ml, dose per injection point of 4.5 U to 5.5 U, volume per injection point of 0.08 ml to 0.13 ml, distance between injection points of 1.8 cm to 2.2 cm, (32) concentration of 40 U/ml to 60 U/ml, dose per injection point of 4.5 U to 5.5 U, volume per injection point of 0.08 ml to 0.12 ml, distance between injection points of 1.8 cm to 2.2 cm, (33) concentration of 40 U/ml to 60 U/ml, dose per injection point of 4.5 U to 5.5 U, volume per injection point of 0.09 ml to 0.11 ml, distance between injection points of 1.8 cm to 2.2 cm, (34) concentration of 50 U/ml, dose per injection point of 4.5 U to 5.5 U, volume per injection point of 0.09 ml to 0.11 ml, distance between injection points of 1.5 cm to 2.5 cm, (35) concentration of 50 U/ml, dose per injection point of 4.5 U to 5.5 U, volume per injection point of 0.09 ml to 0.11 ml, distance between injection points of 1.7 cm to 2.3 cm, (36) concentration of 50 U/ml, dose per injection point of 4.5 U to 5.5 U, volume per injection point of 0.09 ml to 0.11 ml, distance between injection points of 1.8 cm to 2.2 cm, (37) concentration of 40 U/ml to 60 U/ml, dose per injection point of 5.0 U, volume per injection point of 0.083 ml to 0.125 ml, distance between injection points of 1.5 cm to 2.5 cm, (38) concentration of 40 U/ml to 60 U/ml, dose per injection point of 5.0 U, volume per injection point of 0.083 ml to 0.125 ml, distance between injection points of 1.7 cm to 2.3 cm, (39) concentration of 40 U/ml to 60 U/ml, dose per injection point of 5.0 U, volume per injection point of 0.083 ml to 0.125 ml, distance between injection points of 1.8 cm to 2.7 cm, (40) concentration of 50 U/ml, dose per injection point of 5.0 U, volume per injection point of 0.10 ml, distance between injection points of 1.5 cm to 2.5 cm, (41) concentration of 50 U/ml, dose per injection point of 5.0 U, volume per injection point of 0.10 ml, distance between injection points of 1.7 cm to 2.3 cm, (42) concentration of 50 U/ml, dose per injection point of 5.0 U, volume per injection point of 0.10 ml, distance between injection points of 1.8 cm to 2.2 cm, (43) concentration of 50 U/ml, dose per injection point of 5.0 U, volume per injection point of 0.10 ml, distance between injection points of 1.9 cm to 2.1 cm, (44) concentration of 50 U/ml, dose per injection point of 5.0 U, volume per injection point of 0.10 ml, distance between injection points of 2.0 cm.

According to the present invention, the particularly preferred combinations of (1) to (44) can further be combined with a total dose of up to 400 U or 100 U to 400 U or up to 300 U or between 200 U and 300 U or from more than 300 U to 400 U. Also, the particularly preferred combinations of (1) to (44) can further be combined with a total number of injection points of up to 90 or 40 to 90 or 50 to 90 or up to 80 or 50 to 80 or 60 to 80. Furthermore, the particularly preferred combinations of (1) to (44) can further be combined with one of the following combinations: (I) a total dose of up to 400 U and a total number of injection points of up to 90, (II) a total dose of 200 U to 400 U and a total number of injection points of up to 90, (III) a total dose of 200 U to 300 U and a total number of injection points of up to 90, (IV) a total dose of more than 300 U to 400 U and a total number of injection points of up to 90, (V) a total dose of up to 400 U and a total number of injection points of 40 to 90, (VI) a total dose of 200 U to 400 U and a total number of injection points of 40 to 90, (VII) a total dose of 200 U to 300 U and a total number of injection points of 40 to 90, (VIII) a total dose of more than 300 U to 400 U and a total number of injection points of 40 to 90, (IX) a total dose of up to 400 U and a total number of injection points of 50 to 80, (X) a total dose of 200 U to 400 U and a total number of injection points of 50 to 80, (XI) a total dose of 200 U to 300 U and a total number of injection points of 50 to 80, and (XII) a total dose of more than 300 U to 400 U and a total number of injection points of 50 to 80.

The injection points are typically arranged in a grid pattern, preferably in a quadrangular grid, more preferably in a rectangular grid with each of the four injection points forming a rectangle being equally spaced from each other (i.e., there are four equidistant neighbouring injection points for each injection point), and most preferably in a square grid.

Within the present invention, the peripheral neuropathic pain is preferably selected from the group consisting of post-herpetic neuralgia, peripheral nerve injury, trigeminal neuralgia, painful polyneuropathy, and painful radiculopathy. Particularly preferred, the peripheral neuropathic pain is post-herpetic neuralgia, peripheral nerve injury or painful polyneuropathy including diabetic neuropathic pain.

The botulinum toxin used within the present invention is not particularly limited and includes botulinum toxin of any serotype (BoNT/A-H) in a form that is free of complexing proteins or in the form of a complex that contains complexing proteins. Preferably, the botulinum toxin is of serotype A or B (BoNT/A, BoNT/B), with serotype A (BONTA) being particularly preferred. More preferably, the botulinum toxin is of serotype A, still more preferably of serotype A1 (BoNT/A1), and most preferably BoNT/A 1 produced by Clostridium botulinum Hall strain. Preferably, the botulinum toxin is of type A and in a form that is free of complexing proteins or is in the form of a complex that contains complexing proteins and, more preferably, is botulinum toxin of type A in a form that is free of complexing proteins.

As used herein, the term “botulinum toxin” (“BT”) is synonymously used with the term “botulinum neurotoxin” (“BONT”). These terms are intended to refer to a toxin form that is free of complexing proteins, i.e., the (active) neurotoxic polypeptide that ultimately inhibits acetylcholine release (also referred to herein as the “pure botulinum neurotoxin”, “neurotoxic component”, “150 kDa neurotoxin” or “Clostridium botulinum neurotoxin (150 kD)”) and a toxin form that is a complex containing complexing proteins (i.e., a complex of the neurotoxic component and complexing proteins). The botulinum toxin complex is a high-molecular complex of the neurotoxic component and a set of complexing proteins (NAPs), including the 900 kDa, 500 kDa, and 300 kDa C. botulinum type A toxin complexes. The complexing proteins are nontoxic nonhaemagglutinin (NTNHA) and, in strains of serotype A-D, different haemagglutinins (HAS). For example, the 900 kDa complex is included in onabotulinumtoxin A (Botox®/Vistabel®, Allergan, Inc., Irvine, CA, USA), and also abobotulinumtoxin A (Dysport®, Azzalure®, Ipsen, Paris, France), Alluzience® (Ipsen/Galderma) and Innotox® (Medytox) contain a toxin complex as active agent. Preferably, the botulinum toxin is the pure botulinum neurotoxin that is contained in Xeomin® or is Xeomin®, or is the toxin complex contained in Botox® or Dysport® or is Botox® or Dysport®.

Within the present invention, the botulinum toxin may be a natural neurotoxin obtainable from the bacteria Clostridium botulinum or any other botulinum toxin such as a botulinum toxin obtainable from alternative sources, including recombinant technologies and genetic or chemical modification. Chimeric or genetically modified botulinum toxins, i.e., botulinum toxins containing mutations including substitutions, deletions and insertions, are also encompassed by the terms “botulinum toxin”, “neurotoxic component” and the like. Preferably, the mutation does not compromise any of the biological activities of botulinum toxin. However, it is also envisaged to use mutations to modulate the biological activity of the botulinum toxin. Also included are botulinum toxins containing chemically modified amino acids, for example one or more amino acids which are glycosylated, acetylated or otherwise modified, which may be beneficial to the uptake or stability of the toxin. Particularly preferred is the lipidation of the neurotoxic component.

In the context of the present invention, the dose is expressed in biological units because the used botulinum toxin may contain, for example, variable percentages of inactive toxin that contribute to the overall protein load without contributing to efficacy. Within the context of the present invention, the biological potency of botulinum toxin is determined using the mouse bioassay (MBA). The MBA determines the mean lethal dose (LD50) of toxin/neurotoxin after intraperitoneal injection in mice, i.e., the dose of toxin/neurotoxin capable of killing 50% of a group of mice. On this basis, 1 unit (U) of toxin/neurotoxin, as used herein, is defined as one mouse LD 50 (1.0 LD 50=1.0 U). The LD 50 mouse bioassay is the gold standard among various biological, chemical or immunological detection and activity determination methods for botulinum toxin and is known to those skilled in the art (see, e.g., Pearce, L. B.; Borodic, G. E.; First, E. R.; MacCallum, R. D. Measurement of botulinum toxin activity: Evaluation of the lethality assay. Toxicol. Appl. Pharmacol. 1994, 128, 69-77).

Another useful method for determining the biological activity (biological potency) of a botulinum neurotoxin is a cell-based potency assay as it is disclosed, for example, in WO 2009/114748, WO 2013/049508 or WO 2014/207109. The activity results obtained with such cell-based assays correspond to the activity values obtained in the mouse i.p. LD50 assay because the values are calibrated using the LD50 reference standard.

Due to differences in the LD50 tests used by manufacturers of commercial botulinum toxin formulations, the unit potencies indicated by the manufacturers for their commercial botulinum toxin formulations is proprietary and cannot easily be compared. Therefore, within the framework of the present invention, the conversion rates provided below are used to establish the comparative potencies of incobotulinumtoxinA (“INCO”; Xeomin®, Bocouture®; botulinum toxin serotype A, free of complexing proteins; Merz Pharmaceuticals GmbH), onabotulinumtoxinA (“ONA”; Botox®, Vistabel®; botulinum toxin complex of serotype A; Allergan Inc.), abobotulinumtoxinA (“ABO”; Dysport®, Azzalure®; botulinum toxin complex of serotype A; Medicis Pharmaceutical Corp., Galderma Lab.), rimabotulinumtoxinB (“RIM”; Myobloc®, NeuroBloc®; botulinum toxin serotype B; Solstice Neurosciences Inc.), and PurTox® (“TBD”; botulinum toxin serotype A; Mentor Worldwide LLC). For use herein, the conversion rate of ONA and INCO is 1:1. The conversion rate of ONA/INCO: ABO is 1:2.5. The conversion rate of ONA/INCO: RIM is 1:50, and the conversion rate of ONA/INCO:TBD is 1:1.5. Furthermore and preferably, within the context of the present invention, 1 U of INCO (Xeomin®) and 1 U of onabotulinumtoxinA (“ONA”; Botox®) shall be deemed to correspond to one mouse LD 50 (1.0LD 50 ), or 1 U, measured as described above.

Generally, the botulinum toxin used within the present invention is in the form of a liquid composition. The liquid composition can be formulated by various techniques dependent on the desired application, as known in the art. It may be provided as a ready-to-use liquid formulation or in the form of a lyophilized powder that is to be reconstituted, typically in physiological saline, prior to use. Preferably, the botulinum toxin used within the present invention is in the form of an aqueous solution, more preferably a saline solution or a physiological saline solution, and most preferably a phosphate buffered physiological saline solution. The aqueous solution may additionally comprise one or more pharmaceutically acceptable substances. Suitable pharmaceutically acceptable substances comprise those well known in the art, see, e.g., Remington's Pharmaceutical Sciences, Mack Publishing Company, Easton, Pennsylvania.

In particular, the aqueous botulinum toxin solution or composition may include other carriers or non-toxic, non-therapeutic, non-immunogenic stabilizers and the like. Thus, the aqueous botulinum toxin composition may contain glycerol, protein stabilizers (HSA) or non-protein stabilizers such as polyvinyl pyrrolidone (PVP), hyaluronic acid or free amino acids, e.g., methionine or histidine. Suitable non-proteinaceous stabilizers are disclosed in WO 2005/007185 or WO 2006/020208. Also, it may be free of amino acids and/or free of stabilizing peptides (e.g., consisting of 5 to 50 amino acids, 10 to 40 amino acids or 15 to 30 amino acids). The botulinum toxin composition can also include non-ionic or ionic surfactant, e.g., polysorbate or poloxamer. A suitable formulation for HSA-stabilized formulation comprising a botulinum toxin according to the present invention is, for example, disclosed in U.S. Pat. No. 8,398,998B2.

Preferably, the botulinum toxin used within the present invention is in the form of an aqueous solution comprising sodium chloride (NaCl), preferably in the form of a physiological saline solution (i.e. a solution including sodium chloride in physiological concentration, e.g., about 9 g/l NaCl), which further comprises one or more of the following (i) to (ix): (i) no other excipient (except NaCl), (ii) human serum albumin (HSA) and a sugar, in particular a monosaccharide or a disaccharide, (iii) human serum albumin (HSA) and lactose, (iv) human serum albumin (HSA) and sucrose, (v) a monosaccharide and/or a disaccharide (e.g. lactose and/or sucrose), (vi) no buffer, (vii) no single amino acids, (viii) no human serum albumin (HSA), sodium chloride and lactose or no HSA, sodium chloride and sucrose, or (ix) no HSA and sodium chloride, or any combination of (i) to (ix).

Particularly preferred, the botulinum toxin is in the form of an aqueous formulation comprising botulinum toxin, sodium chloride and human serum albumin, or an aqueous formulation comprising botulinum toxin, sodium chloride, human serum albumin and lactose, or an aqueous formulation comprising botulinum toxin, sodium chloride, human serum albumin and sucrose. Another particularly preferred aqueous formulation comprises botulinum toxin, sodium chloride, human serum albumin and histidine. The botulinum toxin can be as defined herein above.

In a second aspect, the present invention relates to a method for the treatment of peripheral neuropathic pain, wherein the method comprises administering an effective amount of botulinum toxin by subcutaneous injection into a skin area of a patient at a concentration of 35 U/ml to 65 U/ml, and at a dose of 4.0 U to 6.0 U per injection point and a volume of 0.07 ml to 0.15 ml per injection point, and wherein the distance between injection points is 1.5 cm to 2.5 cm.

The method according to the second aspect of the present invention is closely related to the use according to the first aspect of the present invention. Thus, all definitions, explanations, advantages etc. given herein with respect to the use according to the first aspect of the present invention equally apply to the method according to the second aspect of the present invention.

### EXAMPLE

### Efficacy and Safety of Botulinum Toxin in the Treatment of Peripheral Neuropathic Pain Using the Dosing and Injection Scheme According to the Present Invention

A parallel-group, placebo-controlled, multicenter, double-blind, randomized two-arm, proof-of-concept clinical study is carried out to investigate the efficacy and safety of subcutaneous injection of botulinum toxin (botulinum neurotoxin or “BONT”), in particular of botulinum toxin type A (BoNT/A), e.g., NT 201 (Xeomin®), in the treatment of peripheral neuropathic pain. The medical condition studied is moderate to severe chronic PNP due to postherpetic neuralgia (PHN) or peripheral nerve injury (postsurgical/post-traumatic neuropathic pain).

A total of, e.g., 120 subjects aged 18 years or older (male or female) are included in the study. The main inclusion criteria include (1) chronic PNP that persists for at least 6 months by the time of the screening visit and is plausibly related to either an episode of herpes zoster or a peripheral nerve injury (caused by surgery or mechanical trauma), (2) documented diagnosis of either chronic neuropathic pain after peripheral nerve injury (i.e., postsurgical/post-traumatic neuropathic pain) or PHN with at least probable level of certainty according to the NeuPSIG/IASP grading system, and (3) a score of at least 4 out of 10 points on the Neuropathic Pain 4 Questions (DN4) questionnaire.

The subjects are randomized with a ratio of 1:1, leading to about 60 subjects in the BoNT group and 60 subjects in the placebo group. At baseline (Day 1), the subjects receive a single intervention session with subcutaneous injections of BoNT or placebo into the PNP skin area. The BoNT formulation used has a concentration of 50 U/ml and can be prepared by reconstitution of, e.g., a 100 U vial of botulinum toxin, for example of NT 201 (Xeomin®), with 2 ml sterile physiological (0.9%) sodium chloride solution, resulting in a concentration of 50 U/ml. The injected BoNT total dose may be up to 400 U and, within this example, is up to 300 U, divided in up to 60 subcutaneous (SC) injections (i.e., 5 U per site). The injections are arranged in a square grid pattern and with a distance of 2 cm between injection points. The subjects of the placebo group receive a placebo solution (0.9% sodium chloride) only containing the excipients of the used saline solution of botulinum toxin (i.e., sucrose and human serum albumin in case of NT 201).

All randomized subjects are then followed for, e.g., a 20-week double-blind follow-up period with daily self-administered clinical outcome assessments (injection session at Day 1). Efficacy variables are determined in the last 7 days before baseline (Day 1) and daily or at regular intervals until the end of the study to assess the change in PNP intensity. The last seven days preceding Day 1 serve as a 7-day baseline pain assessment period. In addition, safety is assessed as known to a person skilled in the art, using safety parameters including Adverse Events (AEs), AEs of Special Interest (AESIs), and serious Adverse Events (SAEs). At the EoS visit after 20 weeks, a global assessment of the overall tolerability of the treatment on a 4-point response scale (1=very good, 2=good, 3=moderate, 4=poor) is performed by the investigator.

The primary efficacy variable is the Average Daily Pain (ADP) at weeks 2 to 12, established by daily self-assessment of the subjects. This variable establishes evidence of efficacy of BoNT in the treatment of PNP over placebo. The pain resulting from chronic neuropathy is the core symptom that directly impacts patients' quality of life. The secondary efficacy variable is the change from baseline in Neuropathic Pain Symptom Inventory (NPSI) total score at Weeks 2 to 12, regularly self-assessed (e.g., every 7 days) by the subjects. This variable is used to estimate the reduction of severity of PNP symptoms in the treatment of PNP over placebo. The assessment of these primary and secondary efficacy variables as well as of other efficacy variables are further explained below.

NRS for ADP, WDP and LDP. Participants will record the PNP intensity of their ADP (average daily pain) as well as their WDP (worst daily pain) and LDP (least daily pain) during the last 24 hours on the Numeric Rating Scale Neuropathic Pain (NRS). Each of the assessments will be rated on an 11-point NRS ranging from ‘0—No pain at all’ to ‘10—Pain as bad as I can imagine’. NRS assessments of ADP intensity are often used as primary efficacy endpoints in clinical pain trials and are recommended by the authorities in the guidance documents “Guideline on the clinical development of medicinal products intended for the treatment of pain” (2016) by the European Medicines Agency (EMA) and “Guidance for Industry Analgesic Indications: Developing Drug and Biological Products (Draft Guidance)” (2014) by the US Food and Drug Administration (FDA).

NPSI. Participants will regularly complete the Neuropathic Pain Symptom Inventory (NPSI), a 12-item questionnaire for the evaluation of typical neuropathic pain symptoms (Bouhassira et al., Pain, 2004, 108 (3):248-57). The NPSI contains ten items asking the participant about the average severity of typical neuropathic pain symptoms such as spontaneous pain (items 1-3), painful attacks (items 5-6), provoked pain (items 8-10), or abnormal sensations (items 11-12) during the past 24 hours. The items are rated on an 11-point scale ranging from 0 indicating no symptoms (e.g., ‘No pain’ or ‘No tingling’) to 10 indicating worst intensity of symptoms imaginable (e.g., ‘Worst pain imaginable’ or ‘Worst tingling imaginable’). The 10 items are used to calculate a total NPSI score as well as five subscores for the different pain domains: (superficial spontaneous) burning pain, (deep spontaneous) pressing pain, paroxysmal pain, evoked pain, paresthesia/dysesthesia. It is also intended to use these ratings for sensory phenotyping. Furthermore, the NPSI contains two items on the duration of spontaneous pain per day (item 4) and the frequency of pain attacks per day (item 7), with five response options each.

PGIC. In the Patient Global Impression of Change PGIC assessment, participants are asked to indicate their overall impression of change since study treatment, using a 9-point Likert scale (from ‘−4—very much worse’ to ‘+4—very much improved’).

EQ-5D-5L and EQ VAS. the EuroQol 5-Dimensions 5-Levels (Eq-5D-5L) documents the health state of participants on the assessment day for the five dimensions of mobility, self-care, usual activities, pain/discomfort, and anxiety/depression (Herdman M, Gudex C, Lloyd A, et al., Development and preliminary testing of the new five-level version of EQ-5D (EQ-5D-5L), Qual. Life Res. 2011, 20 (10):1727-36). For each dimension, participants can choose between five response options indicating no problems, slight problems, moderate problems, severe problems, or extreme problems in each domain. Participants also rate their overall health on the assessment day on the 0 to 100 EuroQol visual analogue scale (EQ-VAS), which ranges from ‘0—The worst health you can imagine’ to ‘100—The best health you can imagine’.

BPI-SF Interference. For the seven interference items of the Brief Pain Inventory—Short Form—Interference (BPI-SF Interference), participants report how pain has interfered with his/her general activity, mood, walking ability, normal work, relations with other people, sleep, and enjoyment of life during the past 24 hours (Cleeland, C. S. and Ryan, K. M., Ann. Acad. Med. Singap., 1994, 23 (2):129-38. The items are rated on an 11-point scale ranging from 0 indicating no interference (‘Does not interfere’) to 10 indicating complete interference (‘Completely interferes’).

HADS. The Hospital Anxiety & Depression Scale (HADS) will be used to quantify anxiety and depression. Each of the two domains is covered by seven items with four response options (Zigmond A. S. and Snaith R. P., Acta Psychiatr. Scand., 1983, 67 (6):361-70). Response options relate closely to the anxiety or depression concept and ask, for example, how often or how much the patient felt that way.

The above-described approach, which involves a combination of injection dose, injection volume, number of injections and distance between injection points, provides a local and minimally invasive alternative to established PNP therapies, which is effective, well tolerated and safe.

1. A method for treating peripheral neuropathic pain comprising administering a botulinum toxin to a patient, wherein the botulinum toxin is subcutaneously injected into a skin area of the patient at a concentration of 35 U/ml to 65 U/ml, and at a dose of 4.0 U to 6.0 U per injection point and a volume of 0.07 ml to 0.15 ml per injection point, and wherein the distance between injection points is 1.5 cm to 2.5 cm.

2. The method according to claim 1, wherein the volume per injection point is 0.08 ml to 0.13 ml.

3. The method according to claim wherein the dose per injection point is 4.5 U to 5.5 U.

4. The method according to claim 1, wherein the concentration is 40 U/ml to 60 U/ml.

5. The method according to claim 4, wherein the dose per injection point is 4.5 U to 5.5 U and the volume per injection point is 0.07 ml to 0.14 ml.

6. The method according to claim 5, wherein the volume per injection point is 0.08 ml to 0.13 ml.

7. The method according to claim 1, wherein the distance between injection points is 1.8 cm to 2.2 cm.

8. The method according to claim 1, wherein the total dose is up to 400 U or the total number of injection points is up to 90, or wherein the total dose is up to 400 U and the total number of injection points is up to 90.

9. The method according to claim 1, according to claim 8, wherein the total dose is between 100 U and 400 U.

10. The method according to claim 1, wherein the injection points are arranged in a grid pattern including a quadrangular grid, a rectangular grid with each of the four injection points forming a rectangle being equally spaced from each other, and a square grid.

11. The method according to claim 1, wherein the peripheral neuropathic pain is selected from the group consisting of post-herpetic neuralgia, peripheral nerve injury, trigeminal neuralgia, painful polyneuropathy, and painful radiculopathy.

12. The method according to claim 11, wherein the peripheral neuropathic pain is post-herpetic neuralgia, peripheral nerve injury or painful polyneuropathy including diabetic neuropathic pain.

13. The method according to claim 1, wherein the botulinum toxin is of type A.

14. The method according to claim 1, wherein the botulinum toxin is in a form that is free of complexing proteins or is in the form of a complex that contains complexing proteins.

15. The method according to claim 1, wherein the total dose is between 200 U and 300 U.

16. The method according to claim 1, wherein the total dose is more than 300 U to 400 U.

17. The method according to claim 1, wherein the total number of injection points is 40 to 90.

18. The method according to claim 1, wherein the total number of injection points is 50 to 80.

19. The method according to claim 1, wherein the total dose is between 100 U and 400 U and the total number of injection points is 40 to 90.

20. The method according to claim 1, wherein the total dose is between 200 U and 300 U and the total number of injection points is 60 to 80.
